(19) United States
(12) Patent Application Publication
(10) Pub. No.: US 2011/0112502 A1
(43) Pub. Date: May 12, 2011

(54) NEEDLELESS DEVICE FOR DELIVERY OF AN AGENT THROUGH A BIOLOGICAL BARRIER

(75) Inventor: Andrew James Kirby, Cardiff (GB)

(73) Assignee: OCELUS LIMITED, Newport (UK)

(21) Appl. No.: 12/673,896

(22) PCT Filed: Oct. 6, 2008
(86) PCT No.: PCT/GB08/50910

§ 371 Date: Feb. 17, 2010

(30) Foreign Application Priority Data
Oct. 8, 2007 (GB) 0719577.9

(51) Int. Cl. A61M 5/30

(52) U.S. Cl. 604/500; 604/57

(57) ABSTRACT

A needleless device (10) for delivery of an agent through a biological barrier, the device comprising a microimplant (12) retaining layer (13) holding at least one microimplant for delivery through the biological barrier, a microimplant driving layer including means (21) for providing a motive force to the or each microimplant, and a guide layer having at least one guide channel associated with a microimplant through which the motive force is applied to the or each microimplant, wherein, in use, activation of the microimplant driving layer causes the or each microimplant to be driven away from the retaining layer through the biological barrier.

The present invention relates to a needleless device that may be placed on a biological barrier, such as the stratum corneum, and then be used to supply an agent through said biological barrier.

Many methods and devices are known for the penetration of biological barriers, including high speed particle penetration (Powderject or Powdermed system), microneedles, liquid jet injection, as well as traditional means such as hypodermic needles. The most widely used of these is the latter—hypodermic needles, a method which is problematic with respect to patient acceptability, pain and frequently requires a skilled healthcare professional. Microneedles are a possible minimally invasive answer to these problems, but after over 30 years of development, fundamental difficulties remain with accurate, clinically adequate and reproducible dosing, manufacturing feasibility and cost. For instance, getting reliable penetration of all holes in the skin from the microneedle array is difficult, and the quality of these holes may differ, decreasing reproducibility. Penetration depth may also be difficult to control. Furthermore, drug formulation must be carefully considered. For instance, coating onto the surface of a microneedle is challenging, and the physical quantity possible by this method is rather small, thereby decreasing dose and therefore limiting the range of drugs deliverable to those of extremely high potency. Other possibilities include injection through holes in the needles themselves, although this is technically challenging, and the epidermis does not provide a good flow-path for injected drugs.

Other needle-free injection systems such as ballistic particle delivery suffer from high complexity and cost, limited portability and difficulty in formulation.

The present invention provides a low-cost, minimally invasive device for the delivery of pharmaceutical or immunological actives or other substances through a biological barrier such as the stratum corneum, allowing a low pain, low skill alternative to conventional needles, with greater reproducibility than microneedles.

In accordance with a first aspect of the present invention, there is provided a needleless device for delivery of an agent through a biological barrier, the device comprising:

a microimplant retaining layer holding at least one microimplant for delivery through the biological barrier;

a microimplant driving layer including means for providing a motive force to the or each microimplant; and

a guide layer having at least one guide channel associated with a microimplant through which the motive force is applied to the or each microimplant,

wherein, in use, activation of the microimplant driving layer causes the or each microimplant to be driven away from the retaining layer through the biological barrier.

The device according to the invention is intended for application to a “biological barrier”. As used herein, the term “biological barrier” refers to any biotic surface that separates a human or animal body from the environment and may include skin (including scalp), eye, mouth-lining, nasal passages, gums, glans penis, external female genitalia, or a wound or incision. The “biological barrier” may comprise epithelial tissue, such the skin and/or mucosa. As used herein, the term “skin” is to given its usual meaning in the art, i.e. the epithelial tissue providing an anatomical barrier between the internal and external environment of the body. The skin is preferably exposed to form the outer surface of the body. The outer-most layer of the skin, that is traversed using a device according to the invention, is known as the stratum corneum. This is the upper most layer of the epidermis; reference to penetrating the skin therefore refers to penetration of at least the stratum corneum.

Delivery of an agent across the skin is referred to in the art as “transdermal delivery”. The device according to the invention is therefore useful for transdermal delivery.

The device according to the invention is useful in both veterinary, i.e. animal health, and medical, i.e. human health applications. The device can further be used in a purely cosmetic method, for example to deliver agents that are intended to improve appearance only, and do not have a therapeutic effect. The invention therefore includes cosmetic methods. Cosmetic agents include, but are not limited to dermal/epidermal fillers such as hyaluronic acid, botulinum toxin or coloured compounds for semi-permanent make-up, tattooing or identification.

The invention is concerned with the delivery of agents across a biological barrier. One embodiment of the invention involves delivery of a vaccine. The term “vaccine” is well known in the art to refer to an agent that is used to establish or improve immunity to a particular disease. A vaccine can be prophylactic or therapeutic and may include conventional and DNA vaccines. In a preferred embodiment, the vaccine is prophylactic to prevent or ameliorate the effects of future infection.

The device may also comprise a biological barrier contact layer, either separate from or integrally formed with the microimplant layer for, in use, contacting the biological barrier.

The biological barrier contact layer may comprise a polymeric film, produced using organic or silicone monomers. Representative examples include, but are not limited to: polyacrylates, polyurethanes, polydimethylsiloxane or other silicones, cellulose derivatives, hydrogels, hydrocolloids, alginates, polyethylene, polyvinylchloride, polyamides, polypropylene, polytetrafluoroethylene or any fluorinated organic or silicone polymer.

Alternatively, or in addition, the contact layer may comprise or contain metallic elements, for example titanium, gold or aluminium film.

The biological barrier contact layer or the microimplant retaining layer may have adhesive on the surface to promote adhesion onto the biological barrier. Representative adhesives for use on the skin include acrylate, hydrogel and silicone adhesives. This may have utility in reducing the ability of the biological barrier to deform under pressure and thereby resist penetration of the microimplants.

The contact layer may be attached to, or may be the same as the layer containing the microimplants. This layer may be formed from the same materials as the contact layer, may be continuous with it, may be identical to it, or may be formed from different materials. Representative examples of material forming this layer include, but are not limited to: polyacrylates, polyurethanes, polydimethylsiloxane or other silicones, cellulose derivatives, hydrogels, hydrocolloids, alginates, polyethylene, polyvinylchloride, polyamides, polypropylene, polytetrafluoroethylene or any fluorinated organic or silicone polymer.

Alternatively, or in addition, the layer may comprise or contain metallic elements, for example titanium or aluminium films.

The microimplants may be practically any shape but preferably they are pointed so as to promote penetration through the biological barrier. Such shapes include cones, pyramids and chisel shapes. The chisel shape may be most advantageous as it may have increased strength in the point to resist breakage. The microimplants may also be shaped so as to promote rotation during penetration, to make a spiral or drilling motion. The microimplants may be polymeric or metallic, or a combination thereof, or alternatively may be crystals or any other substance, for example a drug, or sugars such as maltose. Preferably the microimplants are biodegradeable or bioresorbable or lipid or water soluble and may comprise medical biodegradeable polymers such as but not limited to polylactides, poly (lactic acid-co-glycolic acid), polycaprolates, or polyorthoesters, poly(dioxanone), poly siloxanes, poly(anhydrides), poly(trimethylene carbonate, polyphosphazenes and derivatives or mixtures thereof, or natural polymers such as fibrin, collagen, chitosan, gelatin, hyaluronan, alginates. Alternatively, or in addition, inorganic substances may be used, such as, but not exclusively, calcium hydroxyapatite, or metals such as but not exclusively gold, titanium, steel or alloys of any metal.

The microimplants may be designed to hold a biologically active substance and release it either immediately upon delivery or over a set period of time e.g. 1 week, 1 month, or may comprise a biologically active substance such as immunologicals, biologicals, nucleic acids or cells such as stem cells, or may be biologically inert.

Microimplants may be surface coated, to promote adhesion within the biological barrier, or may change physical or chemical characteristics when in the biological barrier, for example in response to changes in humidity, pH, isotonicity, temperature or any other external factor. In one embodiment, the microimplants are formed from materials that are hard at storage temperatures, and become soft and pliable at physiological temperatures.

In another embodiment, the microimplants comprise a liquid, either aqueous or non-aqueous or an emulsion, or a gel, cream or ointment. Upon pressure supplied from the driving layer, the liquid or gel bursts through the barrier contact layer and is deposited within the biological barrier.

The microimplants are practically any size, from nanometres up to millimetres in height. Most preferably they are between 1 μm and 800 μm in height. They are of practically any density (numbers of implants per cm²), from 1 microimplant per device, to many thousands per cm². The device itself may be any size or shape, for example it may be circular, ovoid, or in a strip shape.

In one preferred embodiment, the microimplants are formed in situ, in depressions within the microimplant containing film itself. The formation of the depressions may be, for instance, through a moulding process around a male master, or in another embodiment, through a hot or cold stamping process onto the film. The master may be produced by a wide variety of processes, including but not limited to micromachining, laser cutting, etching, LIGA, embossing, electroforming, nano-print lithography printing or moulding. The microimplant material is then applied to the microdepression. This may be accomplished in a variety of ways, partly determined by the nature of the material itself. For example, intaglio printing methods can be used, depositing hot melt polymers into the depressions, followed by a doctor blade to remove excess. Alternatively, a squeegie could be used to deposit the material into the depressions, simultaneously removing excess material.

Vacuum, increased or reduced pressure may be used to encourage accurate filling of the microdepressions. The microdepressions may be open-ended or closed ended. The film may be deformed during initial moulding or stamping, or deformed on filling of the microdepressions, or curing, setting or cooling of the microimplants so as to promote better filling or demoulding of the microdepressions. The microdepressions may be coated with a substance, for example a lubricant, to promote better moulding or demoulding, or may be otherwise surface modified.

The surface coating of the depressions in particular might comprise a biologically active agent, for example a vaccine, gene, or any nucleic acid, or might be designed to change the release profile, immunological activity, degradation or pharmacokinetics of a biologically active agent.

Alternatively or in addition, it might be used to enhance penetration of the microimplant into the biological barrier.

The microimplant layer may be modified to facilitate penetration of the microimplants through the microimplant layer. In one embodiment, the area below the microimplant tip is weakened by mechanical or chemical means, or by energy including but not limited to heat, laser, microwave, RF, other electromagnetic energy or forms of radiation such as alpha, beta or gamma irradiation. Alternatively or in addition, the area is weakened during formation of microdepressions. In a further embodiment, at least one cavity is provided in the microimplant layer that are not filled by or approximate to a microimplant, thereby enabling the material to deform as the microimplant is pushed through the layer. Cavities for this purpose may be anywhere in the film, or the film may have bubbles trapped within it.

There may be a further layer over the microimplants, between the microimplant retaining layer and the guide layer. This may be, but is not limited to, a flexible film, or a woven matrix. This layer may be made of any material, representative examples including polymers such as polydimethylsiloxane or other silicones, polycarbonate or other organic polymers, fluorinated derivatives of organic or silicone polymers, metals such as titanium, steel or aluminium. The layer may be directly bonded to or contiguous with the microimplant retaining layer. Alternatively, there may be another layer between these layers, whose functions might include sealing of the microimplant material thereby protecting it from degradation due to environmental or mechanical means, or protection from the contents of the guide layer described below.

In an alternative embodiment, the distal side of the microimplants are attached to the proximal side of the contact layer. The microimplants are therefore in direct contact with the biological barrier. Force from a micropiston or alternative means pushes through the contact layer and pushes the microimplants through the biological barrier.

The guide layer may be formed in a variety of ways, including, but not limited to, moulding, embossing, lithography, stamping, forging, etching, machining, drilling, laser cutting, printing. The guide layer may be of any thickness, but preferably between 30 and 500 μm. The layer can have several functions, including but not limited to, holding micropistons, aligning micropistons above the microimplants, providing a limiting means to stop the penetration of microimplants to below a specific depth (for example using a 500 μm micropiston, with a contact layer thickness of 30 μm and a channel layer thickness of 170 μm the maximum depth of penetration would be limited to 300 μm into the biological barrier). The guide layer may also comprise or contain elements that deform under pressure, then rebound when the pressure is released. This may help to pull the micropistons back to their original position.

The guide layer may comprise a polymeric film, produced using organic or silicone monomers. Representative examples include, but are not limited to: polyacrylates, polyurethanes, polydimethylsiloxane or other silicones, cellulose derivatives, hydrogels, hydrocolloids, alginates, polyethylene, polyvinylchloride, polyamides, polypropylene, polytetrafluoroethylene or any fluorinated organic or silicone polymer. Alternatively, metals may be used.

Alternatively or in addition, the guide layer is used to direct other means of force, such as hydraulic pressure or gas pressure, onto the microimplants in order to urge them through the contact layer and biological barrier. In such an embodiment, a liquid fills the microchannels, with a reservoir above such that applying pressure to the top of the reservoir causes force to be directed down the microchannels and onto the microimplants. The liquid may also contain or comprise at least one biologically active agent, or may comprise or contain a method of sealing any holes in the biological barrier after penetration of the microimplant, or may provide a method of activating, reconstituting or dissolving the microimplant material, or other active agents.

The channels, typically, microchannels, are of any shape, but are preferably slightly tapering.

The microdepressions for the microimplants and the microchannels may be formed at the same time, in such a case, inkjet printing or any other method might be used to deposit material at the bottom of the well, via the channel.

In one preferred embodiment, micropistons are used to push the microimplant through the contact layer and through the biological barrier. Micropistons are preferably smaller in diameter than the channel in the microchannel layers to allow smooth passage through the channel. In one embodiment, they are larger in diameter than the microimplants. The micropistons may be made of any material, preferably organic or silicone polymers or metals such as steel, titanium and aluminium. They must be robust in order to transmit force effectively to the microimplants. Alternatively, the micropistons can be used to apply force to a cavity containing liquid, solid or gas which consequently applies force to the microimplant.

In one embodiment, the micropistons are tapered so as to transmit concentrated force on a smaller microimplant, to facilitate penetration. The micropistons may be independent from each other, may be connected to a flexible upper layer to allow limited independence, or at least one may be connected to one or more others by a solid means. This solid means may be of the same material as the micropistons, or different material.

Having independence, even if limited, is preferable in order to overcome the “bed of nails” effect, resulting in spreading the force applied, thereby making penetration more difficult.

The ability to activate only part of the device, i.e. activate one or more microimplants independently of others, means that dosage can be controlled by applying only part of the deliverable agent at any time. It also means that subsequent doses can be applied using the same device. Alternatively, the independent activation of microimplants could allow extremely precise delivery, particularly if the whole device is transparent, by allowing the user to, for instance, follow the line of a wound or feature like a wrinkle or scar by drawing on the top of the device. The pressure from the drawing could then activate the necessary microimplants into a very specific area of the biological barrier.

In a preferred embodiment, one or more micropistons are pushed sequentially so as concentrate the force applied.

Micropistons may have different diameters to each other. In another embodiment, a single micropiston addresses more than one microimplant at a time. Micropistons also may have different lengths, so as to push microimplants to different depths, or to enable penetration of one part of the device before another part.

In an alternative embodiment, micropistons are moveable to different areas of the device, so that one micropiston can be used to apply force to more than one microimplant, in a serial manner, rather than parallel as described above. The guide layer may be moveable, or alternatively, the micropistons can move from one channel to another channel.

In a further embodiment, microimplants are attached to the proximal side of a layer, with micropistons being embedded at least partially in the contact layer, or a layer approximated to this. Application of force to the micropistons pushes them through this layer and subsequently connects them to the microimplants, pushing them through the biological barrier.

In yet another further embodiment, the micropistons are magnetisable or contain or comprise magnetic material, such that a magnetic force can be used to push the pistons down or retract them.

Micropistons can be formed in a variety of ways, including but not limited to moulding, embossing, extruding, drawing, etching and machining. They may be formed individually, or as a unit of 2 or more connected together. If connected together, the micropistons may be subsequently made wholly or partially independent by modifications such as cutting, machining, weakening, etching, laser cutting of some or all connecting pieces.

The device may further include a means to focus, make sequential, speed up or otherwise apply force. A mechanical applicator may be used either separately or as part of the device to apply mechanical force. In one embodiment, a spring or elastic is used to store and rapidly apply force. In a different embodiment, compressed gas or liquid is the source of the applying force. In a further embodiment, a convex layer is used, snapping into a convave conformation on the application of external force, thereby increasing the speed of application. In a still further embodiment, the breaking of a diaphragm or membrane, or other friable element or attachment is used to increase application speed, for example as in “bubble-wrap” packaging, where sudden breakage of the bubble membrane causes rapid acceleration of the finger.

In an alternative embodiment, a rolling or sliding applicator allows focussing of the force by moving some, but not all of the pistons, below the applicator.

The device may also include control means for determining the distance which the microimplant will be moved through the biological barrier. The same or a different control means may also be provided to control one of more of the dosage applied, the time at which the dose is supplied or the rate at which the dose is applied through the biological barrier. Such a control means may be activated by the patient or by a medical assistant. The controller may have a cut-off that prevents an overdose or prevents the patient getting doses more frequently than desired. The control means may be controlled remotely and or may be operated via a wired or wireless network, or other internet or mobile telephony.

The device could be further provided with a reservoir of drug, either the same or different to the implant drug, which could penetrate the biological barrier either through the holes made by the microimplant process, or through intact skin like a normal transdermal patch. This could give the benefit of a rapid bolus dose, followed by a slower maintenance dose.

The reservoir could be in any of the layers, including the adhesive layer, or could be in another area of the device, for instance feeding into the holes from a liquid reservoir. Alternatively, the product could be a combination device that incorporates both traditional transdermal patch features and the microimplants area in different parts of the patch.

Alternatively, liquid from a reservoir could move through the guide channels in order to activate, reconstitute or promote dissolution of the microimplant material or other actives that are delivered.

The micropistons could be driven through the device using vibration, including ultrasound.

Although the agent to be delivered through the biological barrier is typically located within the guide channels or formed as part of the microimplant itself, in an alternative construction, a further layer is provided in the device, either between the guide layer and the microimplant retaining layer, or, alternatively, between the guide layer and the force providing layer. This additional layer includes the agent to be delivered such that activation of the device causes a portion of that additional layer, including the agent to be delivered to be driven towards the microimplant and subsequently through the biological barrier. Alternatively, the agent can be with the microimplant itself, it can be the microimplant or it can be in a solid rod or other shape behind the microimplant. In addition, as described above, it can be in a separate layer between the microimplant and the guide layer, it can be provided in preformed holes in a separate perforated layer attached to the guide layer, or it can be in a coating on the micropiston or on the mircroimplant itself. The drug could, alternatively, be located in a depressional hole in the microimplant, typically at the rear. The microimplant may be surrounded wholly or partially by agent to be delivered whilst in the microimplant retaining layer such that part of the agent is drawn through the biological barrier when the microimplant is driven. Alternatively the agent to be delivered may be in particles embedded in the microimplant itself.

The device of the present invention can be used in numerous different applications and provided numerous advantages.

One of the advantages is that, especially when the microimplants are retained wholly or partially within the microimplant retaining layer, exposure is reduced to micro-organisms, chemicals or other noxious agents that may be present on the biological barrier. In traditional microneedle applications, there is a significant amount of exposure of the device and the microneedles themselves to the biological barrier, with the resulting danger that noxious agents can be pushed through the biological barrier into the body. A further possibility is that the microimplant or the microimplant retaining layer may be provided with antimicrobial qualities that, should any foreign matter be entrained, the risk of damage is minimised.

The device of the present invention can be used for diagnostic purposes, for instance the delivery of agents such as allegens for patch testing, the response to which allows a physician to determine the presence of absence or course of a disease state. Alternatively, the microimplants can be used to create a pathway from the skin to a device which analysises the body fluid (extra cellular fluid) to determine levels of a chemical, such a glucose. The element behind the microimplant may include a porous or wicked material or, alternatively, a hollow tube or series of tubes.

The material which is delivered through the biological barrier may be for cosmetic or aesthetic purposes such as dermal filler or botulinum toxin, and could include hyaluronic acid, calcium hydroxyapatite, collagen, poly methylmethacrylate, mixtures of all the above, or any other agents to improve texture, appearance, hydration or health of the skin, or to encourage collagen synthesis, skin remodelling or to ameliorate rhytids (wrinkles), promote or prevent hair loss or other facial or skin imperfection or signs of (or course of) aging. It could also include the delivery of genes, growth factors and other biologicals and cells, including stems cells.

Examples of the present invention will now be described with reference to the accompanying drawings, in which

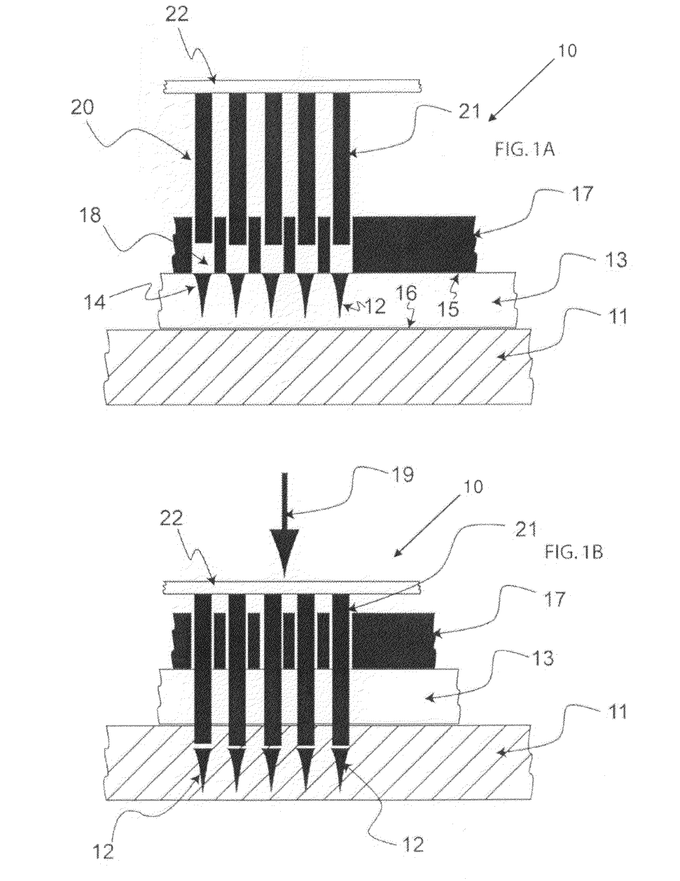

FIGS. 1a and 1b demonstrate a first example;

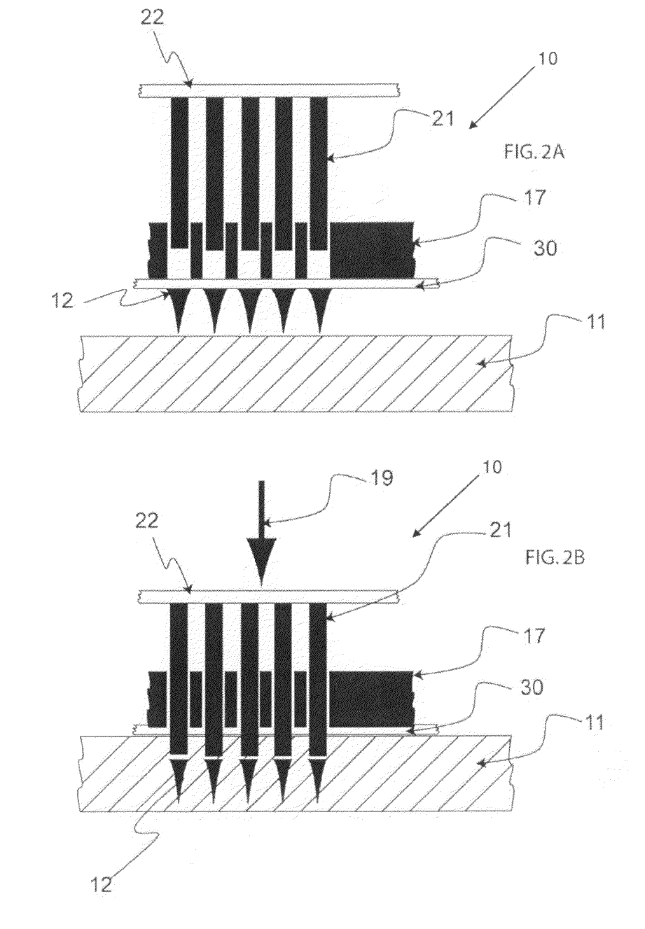

FIGS. 2a and 2b illustrate a second example and

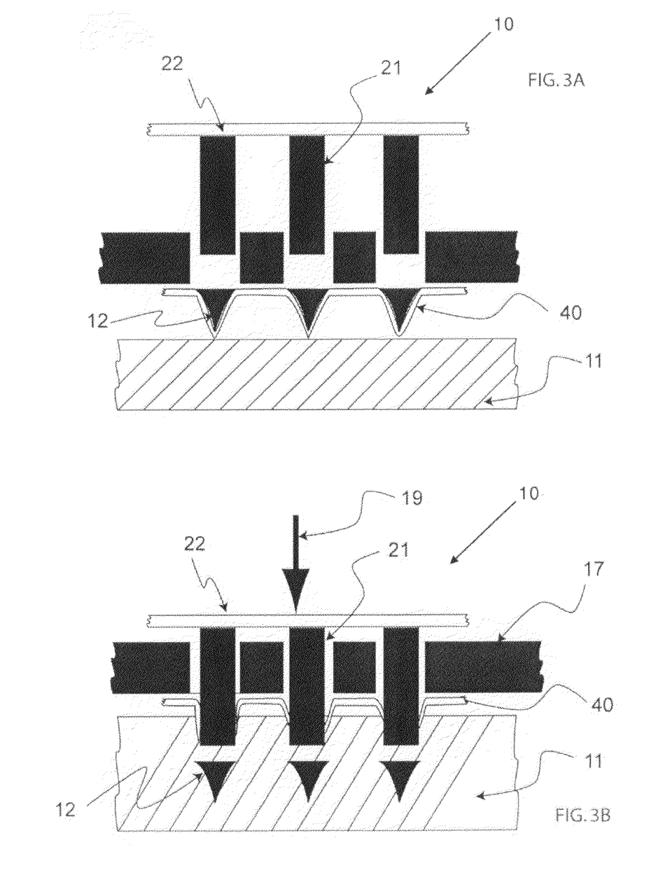

FIGS. 3a and 3b illustrate a third example.

In FIGS. 1a (which shows the device prior to activation) and FIG. 1b (which shows the device after activation) illustrate a device 10 for delivery of an agent through a biological barrier 11. The device is shown as a partial view illustrating only a few microimplants 12. As such, it is not clear that the preferred embodiment is for the device to be self containing, in that the motive means (in this case micropistons) and the agent to be delivered and the microimplants are formed in a unitary product. As shown in the figures, it could be assumed that the motive means (in this case micropistons) were separate from the remainder of the device. This may be possible, but a preferred embodiment would be that all elements described herein are formed within a single housing to provide a self contained device.

Turning now to the figures themselves, the device comprises a microimplant retaining layer 13 in which a plurality of recesses 14 are provided. Those recesses are filled by respective microimplants 12 and this achieved using any of the methods described earlier on this specification. Although shown with the microimplants 12 retained wholly within the layer 13, the implants may be located such that a portion of the upper (in the figures) end of the microimplant protrudes out of the upper surface of the microimplant retaining layer 13. Whilst it may be possible for the lower, pointed end of the microimplant 12 to extend below the lower surface 16, in practice this is undesirable as it is likely that the tip of the microimplants 12 would be damaged or become contaminated. The protrusion of the microimplant above the upper surface 15 may, however, make alignment of additional layers of the device easier.

A guide layer 17 is provided above the microimplants and has a plurality of guide channels 18, each aligned with a respective microimplant 12.

A microimplant driving layer 20 comprising a series of micropistons 21 mounted on a backing layer 22, is provided such that respective micropistons are aligned either with, or in this case within the guide channels 18. The micropistons do not necessarily need to be located within the guide channels, but simply should be aligned such that, upon activation, the micropistons can move through a respective guide channel 18 to contact and drive a microimplant 12 out of the lower surface 16 of the microimplant retaining layer 13.

Whilst it would be usual to have a micropiston associated with each guide channel and a microimplant associated with each guide channel, it may be that, in certain circumstances, additional microimplants, guide channels or micropistons are formed, and therefore it is not essential for there to be the same number of microimplants, guide channels and micropistons.

Upon activation, i.e. by the application of some force or other triggering mechanism, the micropistons are caused to move downwards in the figures, as illustrated by the arrow 19 in FIG. 1b such that the micropistons are driven through the guide channels 18, contact the microimplants 12 which are then driven through the lower surface 16 into the biological barrier 11. As discussed earlier, the microimplants themselves may include the drug formulation which is to be delivered or, alternatively the drug may be located within the guide channel and is driven between the micropistons and the microimplants into the biological layer.

FIGS. 2a and 2b illustrate an alternative construction in which the microimplant retaining layer 12 is replaced by a film 30 on which a series of microimplants 12 are provided. This film 30 may include a drug formulation such that when activated as shown in FIG. 2b, a portion of the film is driven, between the microimplant and the micropiston through the biological barrier. Alternatively, the film may be an inert material which is not harmful.

A further example is shown in FIGS. 3a and 3b, in which, in place of microimplant retaining layer 12, the microimplants are held within depressions in a foil or film layer 40. Again, the film or foil may be an active agent itself or, more likely, it will be an inert material as the pointed microimplant will simply pierce this layer and will not draw any of this film through the biological barrier.

1. A needleless device for delivery of an agent through a biological barrier, the device comprising:
a microimplant retaining layer holding at least one microimplant for delivery through the biological barrier;
a microimplant driving layer including means for providing a motive force to the or each microimplant; and
a guide layer having at least one guide channel associated with a microimplant through which the motive force is applied to the or each microimplant,
wherein, in use, activation of the microimplant driving layer causes the or each microimplant to be driven away from the retaining layer through the biological barrier.

2. A device according to claim 1, wherein the means for providing a motive force is one or more micropistons, wherein each micropiston is preferably associated with a respective microimplant.

3. (canceled)

4. A device according to claim 1, wherein the means for providing a motive force includes a pressurised liquid, gel, or gas supplied through some or all of the guide channels.

5. A device according to claim 1, wherein the or each microimplant is either retained wholly within the microimplant retaining layer or is only partially retained within the microimplant retaining layer.

6. (canceled)

7. A device according to claim 1, further comprising a biological barrier contact layer on the microimplant layer for, in use, contacting the biological barrier.

8. A device according to claim 1, wherein the microimplant retaining layer is formed from silicone.

9. A device according to claim 1, wherein the layer which, in use, is adjacent the biological barrier is provided with an adhesive for attaching to the biological barrier and is formed of at least one of a biodegradable, bioresorbable, bioabsorbable or water or lipid soluble polymer.

10. (canceled)

11. A device according to claim 1, wherein the guide layer is joined to an inner surface of the microimplant retaining layer.

12. A device according to claim 1, wherein the or each microimplant comprises one or more of: a biodegradable, bioresorbable or bioabsorbable polymer, a water or lipid soluble material, an inorganic material, a metal or alloy, a sugar or hyaluronic acid including salts and derivatives thereof, wherein the or each microimplant is formed of a material that changes its mechanical characteristics in response to one or more of: humidity, pH, osmolarity, temperature the presence or absence of water or mechanical, electrical or electromagnetic energy.

13-14. (canceled)

15. A device according to claim 2, wherein, after activation, the or each micropiston moves through a channel in the guide layer.

16. A device according to claim 2, wherein the movement of the micropistons is caused by one of: a pressure activated breakage such as a membrane breaking, a conformational change such as changing from convex to concave, a spring or other biasing means, electromagnetic force or an external mechanical applicator.

17. A device according to claim 2, wherein at least two micropistons or groups of micropistons are provided such that they are movable independently of each other, such that one micropiston or group of micropistons can be moved independent of others, wherein the micropistons are preferably mounted on a flexible membrane or conformable backing layer.

18. A device according to claim 2, wherein the microimplant retaining layer comprises a film or membrane on which the or each micropiston is mounted or comprises a foil material defining recesses in which the microimplants are located.

19. (canceled)

20. A device according to claim 1, wherein a drug, therapeutic agent or other deliverable material is provided in the guide channels such that actuation of the device to drive the microimplant through the biological barrier causes the material provided within the guide channels also to be driven through the biological barrier.

21-22. (canceled)

23. A device according to claim 1, further comprising control means for determining the distance which the microimplants will be moved through the biological barrier, and control means for controlling one or more of the dosage applied, the time at which the dose is supplied, or the rate at which the dose is supplied.

24. (canceled)

25. A method of delivering an agent across a biological barrier, comprising the steps of:
contacting the biological barrier with a device according to claim 1 comprising the agent to be delivered; and activating the means for providing a motive force to the or each microimplant.

26. A method according to claim 25, wherein the agent is a cosmetic agent.

27. Use of a device according to claim 1, to deliver an agent across a biological barrier.

28. Use according to claim 27, wherein the agent is a cosmetic agent or any other agent to improve or modify the aesthetic appearance of the skin.

29. A device according to claim 1, for use in therapy.

30. A device according to claim 29, wherein the therapy is vaccination.
